(19) United States
(12) Patent Application Publication
(10) Pub. No.: US 2013/0303566 A1
(43) Pub. Date: Nov. 14, 2013

(54) METHOD FOR TREATING MACULAR DEGENERATION

(71) Applicant: DAVID W. HILL, Monmouth, ME (US)

(72) Inventor: David W. Hill, Monmouth, ME (US)

(73) Assignee: David W. Hill, Monmouth, ME (US)

(21) Appl. No.: 13/748,816

(22) Filed: Jan. 24, 2013

Related U.S. Application Data
(60) Provisional application No. 61/644,767, filed on May 9, 2012.

(51) Int. Cl. A61K 31/5575; A61K 31/417; A61K 31/472

(52) U.S. Cl. CPC A61K 31/5575; A61K 31/472; A61K 31/417
USPC 514/307

(57) ABSTRACT

The present invention provides a method of treating a mammal with age-related macular degeneration (AMD) comprising administering a dosage of at least one of prostaglandin E1 (PGE1), papaverine, and an alpha-1-vasodilator, in a pharmaceutically acceptable carrier for a period of time effective to deliver a dosage effective to treat said AMD.

### CROSS-REFERENCE TO RELATED APPLICATION

This application claims priority from U.S. provisional patent application Ser. No. 61/644,767, filed May 9, 2012, which is incorporated by reference herein.

### BACKGROUND OF THE INVENTION

Age-related macular degeneration (AMD) is the most common cause of severe and irreversible vision loss in people over 50 years of age. The Macular Degeneration Association estimates that approximately 9.1 million people in the United States older than 40 suffer from AMD. As the population ages, AMD will become an increasing public health concern.

Initially, AMD cases no noticeable symptoms. Once symptom appear, they can include fuzzy or blurred vision, an empty or dark area in the center of the field of vision, the perception of straight lines as curvy or wavy, or a dimming of vision when reading. AMD typically initially presents in one eye, with the likelihood of occurring in another eye increasing over time. Often AMD can lead to blindness, and is the most common cause of legal blindness in Americans 65 and older. The causes of AMD are not entirely known, but are thought to include genetic disposition, diet, smoking, and other factors.

There are two types of AMD—wet AMD and dry AMD. All AMD cases initially present as the dry form, but 10 percent to 15 percent of cases progress to the wet form. Dry AMD can occur in three different stages—early, intermediate, and advanced. Patients with early dry AMD frequently present with small, or few medium sized drusen. Drusen are accumulations of extracellular material that can build up in the eye between the retina and the underlying chloroidal membrane. These drusen can negatively affect a patient's vision. As dry AMD progresses to the intermediate stage, patients may have many medium sized or large drusen causing blurring of vision sensed at that point on the retina. When dry AMD progresses to the advanced stage, light sensitive cells in the macula begin to break down. This occurs principally in the macular part of the retina, causing an opaque gray area in the center of vision.

Wet AMD is more severe than the early and intermediate forms of dry AMD. Wet AMD is often characterized by the growth of abnormal blood vessels under the macula and behind the retina. The new blood vessels can be weak, and leak blood and fluid. The leakage of blood and fluid can cause the macula to swell, causing damage and scarring of the retina. As in the dry type of AMR drusen deposits occur and there is a loss of vision in the macular area. The vision loss experienced by the patient is similar to that caused by dry AMD.

Currently, there is no cure for either form of AMD. Treatment options can include vasodilators, photodynamic therapy, or laser surgery. With wet AMD, abnormally high levels of vascular endothelial growth factor (VEGF) can be secreted in the eye. The secretion of VEGF can serve to promote the growth of new, abnormal blood vessels. The injection of anti-VEGF compounds can reduce macular edema by blocking the harmful effects of VEGF. Complications from the anti-VEGF compounds include eye infections ranging from mild to serious.

The photodynamic therapy technique involves laser treatment of selected retinal areas. For example, the drug verteporfin can be injected intravenously into a patient's arm. The drug travels through the blood vessels in the patient's body, including abnormal leaky blood vessels in the patient's eye. A health care professional can shine a laser beam into the patient's eye to photoactivate the drug within the eye. The activated drug can destroy the new abnormal blood vessels and can slow the rate of vision loss. The drug verteporfin can remain in the patient's body for several days, and can make a person's skin very sensitive to sunburn. Therefore, complications from photodynamic therapy can include severe sunburn. The laser surgery technique is a less common technique employed to treat certain cases of wet AMD. The technique involves aiming an intense beam of light at the new blood vessels in a patient's eye to destroy the new blood vessels. However, laser treatment also may destroy some surrounding healthy tissue and can cause an increase in blurred vision.

It has been reported that vasodilators can be used to treat AMD. In the 1950s, over 300 patients were treated in a 13 year period with a combination of four vasodilators. See, e.g., H. Wyatt Laws, Peripheral Vasodilators in the Treatment of Macular Degenerative Changes in the Eye, Can Med Assoc J. 1964; 91(7):325-330. Others have also postulated that vasodilators might provide useful treatment for AMD. ACE-inhibitors and AR-blockers are examples of vasodilators suggested as possible treatment for circulatory problems including AMD. See, e.g., T. Fisher, New possibilities in the pharmacologic prevention of age-related macular degeneration, Orvosi Hetilap, 2008 149(3):121-7.

A small clinical trial was conducted by UBS, Inc., under Clinical Trials.gov. identifier NCT00619229, in which 15 patients with dry AMD received PGE1 infusions. Patients were given 60 meg of PEG1 in the form of alprostadil by intravenous infusion daily for 15 days. However, the trial was discontinued due to lack of statistically significant results. See, also. M. S. Ladewig, et al,. Prostaglandin E1 infusion therapy in dry age-related macular degeneration, Prostaglandins Leukot Essent Fatty Acids, 2005, 72(4):251-6 and H. Heinrich et al., A pilot study. Klin Monbl Augenheilkd, 1989, 194(4);282-4.

AMD frequently progresses even with treatment. The loss of central vision can happen quickly. Current therapies only attempt to delay the progression of AMD. Therefore, a continuing need exists to develop an effective treatment of AMD.

### SUMMARY OF THE INVENTION

The present invention provides a method of treating a mammal, such as a human, afflicted with age-related macular degeneration (AMD). In one aspect of the invention, the method can include administering a treatment by subcutaneous injection of about 1-8 mcgs per dose of prostaglandin E1 (PGE1), about 1.5-12 mgs per dose of the vasodilator papaverine, about 0.1-0.5 mgs per dose of an alpha-1-vasodilator, such as phentolamine, or a combination thereof (the “active ingredients”). At least one of the PGE1, the papaverine or the alpha-1-vasodilator can be administered, as by injection, in a pharmaceutically acceptable carrier for a period offline effective to deliver a dosage of one or more of the active ingredients effective to treat the AMD.

In one embodiment of the invention, the method of treating a human inflicted with AMD can include the administration of PGE1 (“alprostadil”). In accordance with one aspect of the invention, the daily administration of PGE1 can be in the amount of about 1-3 mcg per single or multiple doses. In one specific embodiment, the PGE1 can be Caveject® (Pfizer) which is injected after reconstitution with sterile water or isotonic saline.

In one specific embodiment of the invention, the method of treating a human inflicted with AMD can include the administration of papaverine including its salts such as papaverine hydrochloride. In one aspect of the invention, the daily administration of papaverine can be in the amount of about 1.5-4.5 mg per single or multiple doses, optionally in a pharmaceutically acceptable carrier.

In one embodiment of the invention, the method of treating a human inflicted with AMD can include the subcutaneous injection of about 0.1-0.8 mg per dose of a second vasodilator, preferably an alpha-1-vasodilator. In accordance with one aspect of the invention, the alpha-1 -vasodilator is phentolamine, including the salts thereof, such as phentolamine mesylate. In a specific embodiment, the alpha-1-vasodilator is administered daily. In a more specific embodiment, the daily administration of the alpha-1-vasodilator is in the amount of about 0.1-0.3 mg per single or multiple doses, in a pharmaceutically acceptable carrier, in one aspect of the invention, the administration of the PGE1, papaverine, and the alpha-1-vasodilator is simultaneous in time. In accordance with one specific embodiment, the PGE1, papaverine, and the alpha-1-vasodilator are injected in a single unit dosage form. Phentolamine is available, as OraVerse® for injection.

In one aspect of the invention, the administration of at least two of the PGE1, the papaverine, and the alpha-1-vasodilator, e.g., the phentolamine, are administered in combination, is simultaneous in time. In one embodiment, the PGE1 and the papaverine are administered in combination, in a pharmaceutically acceptable carrier. In one embodiment, the daily administration occurs for a period of time of at least about 3 weeks.

In one aspect of the invention, the human in need of treatment is at least about 50 years old in age. In one embodiment of the invention, the method of treatment is administered to a human afflicted with AMD, who has not yet progressed to macular blindness. In one aspect of the invention, the AMD is the dry form.

In one aspect of the invention, a method of treating a mammal with dry age-related macular degeneration (AMD), includes administering to the mammal PGE1 and papaverine in a pharmaceutically acceptable carrier by subcutaneous injection, wherein the administration is carried out on a daily basis for a period of time to deliver a dosage of PGE1 and the vasodilator in an amount effective to treat said AMD. In accordance with one embodiment, the method can further include subcutaneous injection of phentolamine, either singly or in combination with the PGE1 and papaverine, in a pharmaceutically acceptable carrier.

### BRIEF DESCRIPTION OF THE FIGURES

In the figures, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 Chart of visual acuity over last five years of subject's OS showing all data obtained during eye examinations, wherein the numbers indicate the following: 1. failed driver's license examination, 2. cataract surgery OS, 3. trial period began, 4. injections, 5. treatment fur anal carcinoma began and 6. cancer radiation and chemotherapy ended.

FIGS. 2 and 3 depicts retinal photographs taken of the eyes of a patient with AMD in December 2011 and December 2012, respectively, FIGS. 1A and 2A depict the right eye of a patient with wet AMD. FIGS. 1B and 2B depict the left eye of a patient with dry AMD. White flecks are drusen deposits.

### DETAILED DESCRIPTION

### Definitions

Unless stated otherwise, the following terms and phrases as used herein are intended to have the following meanings:

AMD is a condition characterized by degeneration of the macula, which is part of the retina responsible for central vision. AMD is also often associated with the presence of β-amyloid containing “drusen deposits.” As used herein, “age-related macular degeneration (AMD)” refers to degeneration of the macula occurring in patients older than 50. AMD can be diagnosed as wet or dry. As used herein, “dry age-related macular degeneration (AMD)” refers to non-neovascular AMD, which is macular degeneration not accompanied by an abnormal growth of new blood vessels in an eye. As used herein, “wet age-related macular degeneration (AMD)” refers to neovascular AMD, which is macular degeneration that is accompanied by an abnormal growth of new blood vessels in an eye.

The phrase “pharmaceutically acceptable” refers to those compounds, materials, carriers, vehicles, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

“Therapeutically effective amount” is intended to include an amount of a combination of active compounds described herein, e.g., to treat or prevent the disease or disorder, or to treat the symptoms of the disease or disorder, in a host. The combination of compounds preferably gives rise to a synergistic result. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial of the combination compared with the individual components.

As used herein, “treating” or “treat” includes (1) preventing a pathologic condition from occurring (e.g., prophylaxis); (ii) inhibiting the progression of the pathologic condition or arresting its development; (iii) relieving the pathologic condition; and/or (iv) diminishing at least one symptom associated with the pathologic condition.

An effective treatment schedule described herein can include a daily injection of a low dose of a prostaglandin E1 (PGE1) in a pharmaceutically acceptable liquid carrier. The treatment schedule can include a daily injection of the vasodilator papaverine in a pharmaceutically acceptable liquid carrier. The treatment schedule can also include a daily injection of an alpha-1-vasodilator such as phentolamine in a pharmaceutically acceptable liquid carrier. The PGE1, papaverine and phentolamine can be administered singly, or in any combination of two active ingredients, or all three can be administered together, e.g. in combination in a single carrier as by separate injections (2 or 3). Acceptable liquid carriers can include isotonic aqueous saline, which can include effective solubilizing and/or stabilizing amounts of alcohols, such as ethanol, and one or more preservatives.

As used herein, “TriMix,” refers to a mix of prostaglandin E1 (PGE1), an alpha-1-vasodilator and a second vasodilator, in a pharmaceutically acceptable carrier. Typically, commercially available TriMix contains alprostadil, phentolamine, and papaverine. For example, 10 ml TriMix for injection can contain 200 μg PGE, 300 mg of papaverine hydrochloride and 20 mg of phentolamine mesylate in sterile water containing a minor but effective amount of ethanol as a co-solvent (e.g., 1-5 wt-% ethanol) and a minor but effective amount of benzyl alcohol as a preservative (e.g. about 0.01 ml.). For example, a preferred unit dosage form is about 0.05-0.25 ml TriMix, e.g., about 0.05 to about 0.1 ml, which can deliver about 1-2 mcg PGE1, about 1.5-3.0 mg papaverine and about 0.1-0.2 mg phentoamine.

References in the specification to “one embodiment,” “an embodiment,” “an example embodiment,” “an example,” etc., indicate that the embodiment described may include a particular feature, structure, or characteristic, but every embodiment may not necessarily include the particular feature. structure, or characteristic. Moreover, such phrases are not necessarily referring to the same embodiment. Further, when a particular feature, structure, or characteristic, is described in connection with an embodiment, it is submitted that it is within the knowledge of one skilled m the art to affect such feature, structure, or characteristic in connection with other embodiments whether or not explicitly described.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The PGE1 or Alprostadil is commercially available from Pfizer and is (11α,13E,15S)-11,15-Dihydroxy-9-osoprost-13-en-1-oic acid or 3-hydroxy-2-(3-hydroxy-1-octenyl)-5-oxocyclopentancheptanoic acid. The amount of PGE1 injected can be approximately 8 mcg, 7 mcg, 6 mcg, 5 mcg, 4 mcg, 3 mcg, 2 mcg, 1 mcg, or less than one meg, e.g., 0.25-0.5 mcg.

Alternatively, or in combination with PGE1 and/or an alpha-1-vasodilator, the present method can include papaverine, including the pharmaceutically acceptable salts thereof, or other phosphodiesterase inhibitors that have vasodilation activity. Papaverine is commercially available from Sandoz and is 1-[(3,4-Dimethoxyphenyl)-methyl]-6,7-dimethoxyisoquinoline or 6,7-dimethoxy-1-veratrylisoquinoline. The amount of the vasodilator drug, such as papaverine, injected, can be approximately 30 mgs, 25 mg, 20 mgs, 15 mgs, 12 mgs, 10 mgs, 5 mgs, 4.5 mgs, 4 mgs, 3.5 mg, 3 mgs, 2.5 mgs, 2 mgs, 1.5 mgs, 1 mg, or less than 1 mg.

Alternatively, or in combination with papaverine and/or PGE1. The present treatment can comprise an alpha-1-vasodilator. The alpha-1-vasodilator can be phentolamine, including pharmaceutically acceptable salts thereof, or any other suitable alpha-1-vasodilator. The alpha-1-vasodilator can be phentolamine mesylate. Phentolamine is commercially available from Novartis AG and is 3-[[(4,5-Dihydro-1H-imidazol-2-ylmethyl](4-methylphenyl)aminp]phenol, 2[N-(m-hydroxyphenyl)-p-toluidinomethyl)]imidazoline, 2-(m-hydroxy-N-p-toluidinomethyl)-2-imidazoline, or 2-(N′-p-tolyl-N′-m-hydroxyphentylaminomethyl)-2-imidazoline. The amount of alpha-1-vasodilator injected can be approximately 2 mgs, 1.5 mgs, 1 mg, 0.9 mg, 0.8 mg, 0.7 mg, 0.6 mg, 0.5 mg, 0.4 mg, 0.3 mg, 0.2 mg, 0.1 mg, or less than 0.1 mg.

The administration of one, any two or all three active ingredients can be carried out on a daily basis, or every other day, or 2-4× week. Any of the disclosed agents can be administered singly. The administration of any of the disclosed agents can occur simultaneously with another disclosed agent in one single dose. The administration of any of the disclosed agents can occur consecutively in time. As used herein, “consecutively in time” can mean that the administration of one drug occurs after the administration of the other, or the administration of a second drug can overlap with the administration of the first. The administration can be carried out every other day, every two days, or every three days. e.g., preferably for 3, 4, 5, 6 or 7 days/week, wherein the 3, 4, 5 or 6 day schedules can be consecutive or spaced apart, or both. The administration can occur for a period of years, for at least a year, for at least 11 months, for at least 10 months, for at least 9 months, for at least 8 months, for at least 7 months, for at least 6 months, at least 5 months, for at least 4 months, for at least 3 months, for at least 2 months, or for at least month. The administration can be interrupted and resumed as needed.

### Routes of Administration

Suitable routes of administration include oral in the case of agents that are orally active, such as papaverine, phentolamine or doxazosin. Parenteral administration is generally preferred, including rectal, nasal, topical (including eye drops, buccal and sublingual), vaginal and by subcutaneous, intraperitoneal (i.p.) intramuscular, intradermal, intraocular, intravitreal, intrathecal and epidural infusion or injection or stereotactic neurosurgical intracranial injection, and the like. Transdermal administration via films, bandage, depots, contact lenses or patches is also useful in some cases. It will be appreciated that the preferred route may vary with for example the condition of the recipient.

### Combination Therapy

The combination of alprostadil, papaverine, and phentolamine can also be used alone, or in combination with other active ingredients. In either embodiment, the specific combination is selected based on the AMD to be treated, cross-reactivities of ingredients and pharmaco-properties of the combination.

The combination of alprostadil, papaverine, and phentolamine can also be used alone, or in combination with other active ingredients, in a unitary dosage form for simultaneous or sequential administration to a patient. In either embodiment, the combination therapy may be administered as a simultaneous or sequential regimen. When administered sequentially, the combination may be administered in two, three or more administrations.

The combination therapy may provide “synergy” and “synergistic effect,” i.e., the effect achieved when the active ingredients used together is greater than the sum of the additive effects that results from using the compounds separately. A synergistic effect may be attained when the active ingredients are: (1) co-formulated and administered or delivered simultaneously in a combined formulation; (2) delivered by alternation or in parallel as separate formulations; or (3 ) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. In either case, an effective therapeutic amount of each agent is present in vivo simultaneously.

Pharmaceutical kits useful in the present invention, which include a therapeutically effective amount of a pharmaceutical composition that includes (a) alprostadil, (b) papaverine, and (c) phentolamine, in one or more sterile containers, are also within the ambit of the present invention. Sterilization of the container may be carried out using conventional sterilization methodology well known to those skilled in the art. Component (a) and component (b) may be in the same sterile container or in separate sterile containers. The sterile containers or materials may include separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, a syringe, a preloaded syringe, etc., as will be readily apparent to those skilled in the art. Instructions, either as inserts or as labels, indicating quantities of the components to be administered, guidelines for administration, and/or guidelines for mixing the components, may also be included in the kit.

The invention will be further described by reference to the following detailed example.

### Example 1

### I. Introduction

A man, now 83 years old, developed AMD in his right eye and later developed dry AMD in his left eye. The patient's vision in his left eye (OS) had begun to deteriorate rapidly, leaving the patient with the expectation of being blind for the rest of his life. The patient experienced wet AMD leakage in his right eye (OD) in 1886. The patients eye was sealed in the area of leakage with 500 shots from a low-powered laser. For two to three years, vision in that eye remained at 20/20 until chloroidal neovascularization of the macula gradually blocked all central vision. The patient is blind in that eye except for peripheral vision.

The patient experienced dry AMD in his left eye. The dry AMD progressed slowly, until the patient noticed a rapid darkening of vision in 2008. He reported experiencing deformed and blurry vision, and could not pass the vision test, 20/40, for a driver's license.

### II. Treatment Procedures

### A. Materials.

To test the hypothesis that vasodilators can successfully treat AMD, TriMix injectable blend was obtained containing, in 10 ml of water, 0.4 ml ethanol, 0.3 g papaverine hydrochloride, 0.02 g phentolamine mesylate and 200 mcg prostaglandin. E1 (PGE1 or “alprostadil”), with 0.01 ml benzyl alcohol. Each of these drugs is commercially available. TriMix can be ordered online from trimixinjection.com. The following example explores the role of administering Trimix to a patient with AMD. The results show that a low daily dose of TriMix containing a low dose of a PGE1 and vasodilators can provide benefits to a patient in need thereof.

Daily subcutaneous injections of TriMix were given into the patient's midriff with an insulin type needle beginning in October 2010. Initial doses were 1 mcg alprostadil, but were upped to 2 mcg of alprostadil for a period of time and then lowered to the original dose. Thus, on a daily basis, 0.85-0.10 mL of the above TriMix blend can be injected into the patient, delivering 1-2 mcg alprostadil, 0.1-0.2 mg phentolamine and 1.5-3.0 mg of papaverine. Doses as high as 8 mcg of PGE1 were also tested without any problems. The patient reported no pain and no side effects except for minor drug interactions. The injections were stopped in May 2012, due to cancer therapy, and resumed in November 2012, by injecting a daily dose of 0.5-1.0 μg PGE1 alone in 0.05-0.25 ml of water. The injections were discontinued in January 2013.

### B. Results.

The patient reported experiencing beneficial results within about thirty minutes of injecting the formulation. The patient reported brighter vision with some scintillation occurring in the macular area of the retina where damage had occurred. The patient has reported no further damage from AMD, and has reported improvements in vision. The patient reported brighter vision with fewer dark or blank patches, improved acuity and an improvement in color blindness. The patient reported recovering a considerable portion of vision in his right eye. The patient reported a smaller opaque spot and further reported being able to see light, large objects and color through the center of his vision. The patient reported distortions in his left eye.

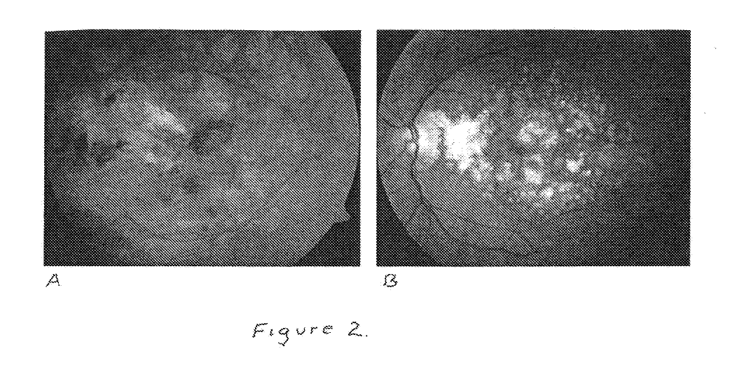

FIG. 2 shows Fluorescein Angiogram retinal photographs taken of the patient's eyes approximately 13 months into treatment (December 2011). FIG. 2A shows the patient's right eye. Dark spots can be seen in the right eye, which are sears resulting from laser treatment of wet AMD. FIG. 2B shows the patient's left eye. Notably, drusen deposits can be seen in the macula and surrounding area of the left eye.

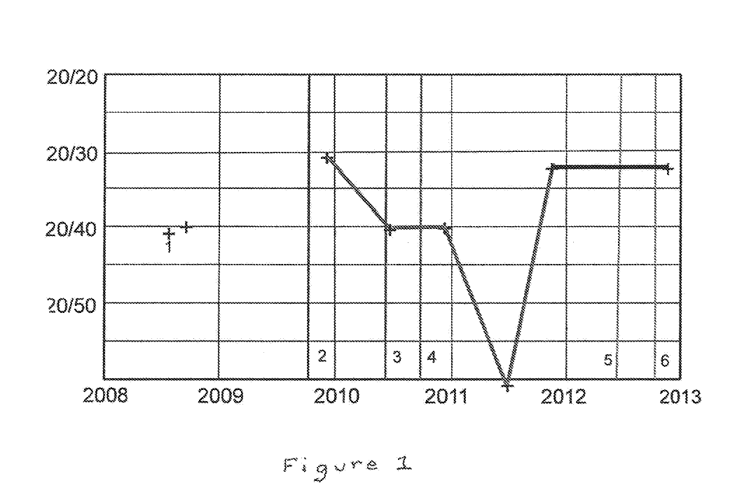

In December 2011, an eye examination by a retinal specialist found no leakage from wet AMD in either eye, and no problems caused by the TriMix medication. The examination confirmed an improvement in visual acuity from 20/60 to 20/32, as shown in FIG. 1. The condition of OS and OD is shown in FIG. 2.

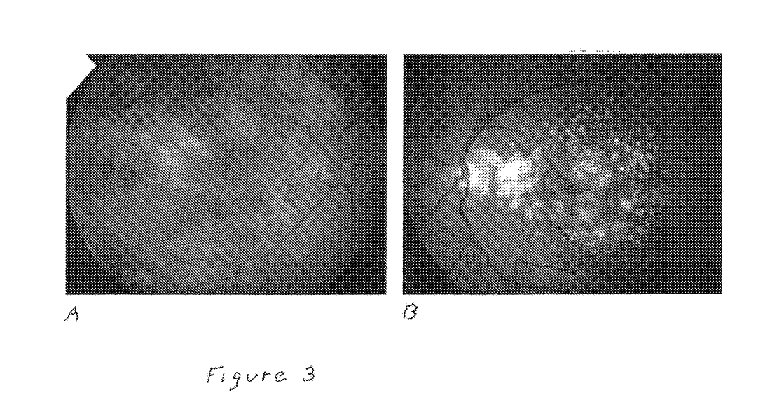

In December an eye examination by a retinal specialist found no leakage from wet AMD in either eye, no evidence of current degenerative activity and no apparent damage since the examination in 2011, as shown in FIG. 3.

FIG. 1 shows a chart displaying the recent history of visual acuity of the patient's left eye as reported by a practicing eye care physician during routine exams. The patient reported an increase in visual acuity following cataract surgery, which was followed by an initial decline after starting medication.

Since receiving combination therapy, the patient has reported an increase in intuitive perception and vision measures have continued to be positive. The patient reported that prior to therapy, he could not see any of six Ishihara Color Blindness Tests clearly, although he did report being able to make out a bit of chart A. Within twelve months of receiving therapy, the patient repotted that Chart A became visible. Two months later, Chart E became visible, and within three months (February 2012), Chart D became visible to the patient. The patient now reports that he is beginning to see two others. The patient reports that color blindness has decreased in OS over the course of his therapy.

In summary, these results demonstrate that a low daily dose of PGE1, papaverine and phentolamine (“TriMix”). can provide benefits to a patient with AMD. As demonstrated by FIG. 1, for some patients, daily administration of TriMix will he needed in order to achieve effectiveness. After a single dose some patients may begin to notice beneficial results immediately. However, in some cases, daily administration is required to achieve stable effects. These results indicate that for some, daily administration of TriMix is indicated until a total dose of 300-400 mcg PGE1 is achieved. An effective treatment regimen can comprise an amount of about 1.0 mcg PGE1 given alone or in TriMix daily for 4 weeks following by about 1.0 mcg doses of PGE, daily for 4-6 months. Treatment can be stopped at this point to observe if further treatment is necessary to at least stabilize vision. In some cases, progress using the treatment described herein can take more than one year to reach the 20/20 level. In some cases, the treatment can last two years or more or others, the treatment can last several years to achieve acceptable results.

Non-steroidal anti-inflammatory drugs (NSAIDs) such as aspirin, acetaminophen and naproxen sodium cause the mask of the damaged macular area to appear colored, such as bright red, can cause misty vision, and should be avoided. Linosopril and other ACE inhibitors can have similar deleterious effects, and their use with TriMix to regulate blood pressure is not preferred.

Patients with newly diagnosed AMD and those with more advanced AMD where macular blindness has not yet occurred may benefit most from this treatment. The treatment described herein can provide a much needed therapy for AMD by Halting or inhibiting the progress of AMD.

### ADDITIONAL NOTES

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 CFR. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to au claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should he determined with reference to the appended claims, along with the full scope of equivalents to which such claims are entitled.

1. A method of treating a mammal with age-related macular degeneration (AMD) comprising administering to a mammal afflicted with AMD, by subcutaneous injection, an effective amount of up to about 1.0 mg per dose of prostaglandin E1 (PGE1), an effective amount of up to about 1.5 mg per dose of papaverine, and an effective amount of up to about 0.2 mg per dose of phentolamine, in a pharmaceutically acceptable carrier, for a period of time effective to treat said AMD.

2.-3. (canceled)

4. The method of claim 1, comprising administering an effective amount of up to about 0.1 mg per dose of phentolamine.

5. (canceled)

6. The method of claim 3 wherein one dose of the PGE1, the papaverine and the phentolamine is administered daily.

7. The method of claim 6, wherein the PGE1, the papaverine, and the alpha-1-vasodilator are injected in a single unit dosage form.

8. (canceled)

9. The method of claim 7, wherein the unit dosage form is administered daily.

10.-12. (canceled)

13. The method of claim 1 or 4, wherein the administration occurs For a period time of at least about 3 weeks.

14. The method of claim 1, wherein the mammal is a human.

15. The method of claim 14, wherein the human of at least about 50 years old in age.

16. The method of claim 1, wherein the AMD has not yet progressed to macular blindness.

17. The method of claim 1, wherein the age-related macular degeneration is the dry form.

18.-19. (canceled)

20. The method of claim 1, wherein the PGE1, the papaverine and the phentolamine are injected in water or isotonic saline that further comprises a minor but effective amount of ethanol.

21. The method of claim 20, wherein the water or isotonic saline comprises about 2.5-7.5 wt-% ethanol.

22. The method of claim 21, wherein the water or isotonic saline further comprises a minor but effective amount of a preservative.

23. The method of claim 1, wherein NSAIDs are not administered to the mammal.
